(19) United States
(12) Patent Application Publication
(10) Pub. No.: US 2009/0062770 A1
(43) Pub. Date: Mar. 5, 2009

(54) SEALING STOPPER AND ASSEMBLY COMPRISING SUCH A SEALING STOPPER

(75) Inventors: Niek EXALTO, Aerdenhout (NL); Mark Hans Emanuel, Aerdenhout (NL)

Correspondence Address: YOUNG & THOMPSON, 209 Madison Street, Suite 500, ALEXANDRIA, VA, 22314, US

(73) Assignee: GYNAECOLOGIQ, ROTTERDAM (NL)

(21) Appl. No.: 12/044,342

(22) Filed: Mar. 7, 2008

Related U.S. Application Data
(63) Continuation of application No. PCT/NL2005/000646, filed on Sep. 7, 2005.

(51) Int. Cl. A61M 39/02; A61M 39/22

(52) U.S. Cl. 604/515; 604/247; 604/278

(57) ABSTRACT

In order to enlarge the uterus, inter alia for diagnostic purposes, it is proposed to seal the cervix once a liquid, such as a gel, has been introduced, by means of a sealing stopper, so that liquid does not constantly have to be supplied to the uterus. The sealing stopper is preferably coupled to a gel feed device which may comprise a simple syringe-type structure. This system comprises a non-return valve, as a result of which a gel can only be displaced in the direction of the uterus. In order to ensure that the sealing stopper can be removed, return means, such as a piece of string, are attached thereto.

### BACKGROUND OF THE INVENTION

In conducting diagnostic treatments such as echoscopy, it is usual that the uterus is enlarged by introducing a watery liquid. This is carried out with a hose which is arranged in the cervix. The hose is fitted with a stopper which abuts the external ostium. During echoscopy the flow of water is constantly monitored and at the location of the stopper water constantly escapes from the hose while the water is being introduced via the hose and by accurately controlling the amount of water supplied, the enlargement of the uterus can be controlled.

Such an enlargement method is uncomfortable for the individual being examined and inconvenient for the treating physician. After all, the uterus has to be monitored constantly in order to ensure that the enlargement remains unchanged. With echoscopy in particular, it is important that no changes occur since the images obtained by this method otherwise do not allow unambiguous diagnosis or treatment.

In addition, this method requires the introduction of water in the location where the echoscopy is carried out. This means that a gynecologist has to be present in order to introduce the water at the point in time when the respective treatment is being carried out. Not only does this lead to increased costs, but it is also very inefficient, since the gynecologist does riot always work at the same site where echoscopies are carried out.

When using a balloon catheter, it is difficult for the treating physician to judge when sufficient engagement between the catheter and the respective organ takes place. Therefore, the physician will tend to opt for an excessive inflation pressure, which is very uncomfortable for the individual in question.

### PRIOR ART

From U.S. Pat. No. 5,248,304 a sealing stopper is known consisting of an inflatable balloon catheter inside which a tube for introducing a fluid can be arranged.

With the device according to U.S. Pat. No. 5,248,304, it is not readily possible to move the individual to be examined once the stopper has been introduced.

U.S. Pat. No. 3,312,215 discloses a multipurpose utero cervical cannula that could be entered in the cervix and extend into the uterus. A conduit having non return valve flaps is provided to take care for uterine drainage and arresting the flow of liquid from the vagina into the uterus. For some treatments wherein the presence of gelatin is required, a gelatin capsule is placed on the tip of the device to be entered in the human body.

EP 0,891,757 discloses a sterilization device to be entered in the fallopian tube.

U.S. Pat. No. 2,392,045 discloses a device for the occlusion of fallopian tube.

### SUMMARY OF THE INVENTION

It is an object of the present invention to achieve the enlargement of the uterus in a more simple manner.

It is a further object of the present invention to provide a more efficient treatment of patients both with regard to the patient himself and the medical personal involved.

According to the present invention, a sealing stopper can be used which is dimensioned such that it can be introduced in the cervix and provides a seal between the internal ostium and the external ostium. In principle, this seal is completely tight so that it is not necessary to provide a certain quantity of liquid in compensation for any liquid leaking away. This means that the individual to be treated/examined can be treated at an outpatients' department, for example, in order to temporarily enlarge the uterus and can subsequently receive further treatment, for which enlargement of the uterus is desirable, at another location. At this other location, the presence of a gynecologist is no longer required, resulting in appreciable improvements in efficiency. Moreover, it will be understood that since no water is leaking away, the individual to be treated/examined is more comfortable, and the medic in question can carry out his work more easily.

This perfect seal is achieved by providing a number of flexible ribs in the longitudinal direction of the core, extending radially outward therefrom, which flexible ribs are spaced apart. More in particular, these flexible ribs are preferably of circular design. More in particular, the diameter thereof becomes smaller toward the free end of the core.

The external shape of the ribs may be such that, on the one hand, optimum engagement is ensured and, on the other hand, minimal inconvenience for the individual to be examined is caused. Examples of the engagement surface of the ribs with the respective organ which may be mentioned include designing them as foam-like parts or cylindrical. It is also possible for the ribs not to extend radially.

According to a particular preferred embodiment, however, the ribs are designed as a number of spaced apart spherical parts, through which the (hollow) core extends. The diameter of these spherical parts may become smaller in the direction of the free end of the core. By way of example, an embodiment comprising three consecutive spheres with a diameter of, for example, 4.5 and 6 mm is mentioned. It will be understood that, depending on the organ, into which the device is to be introduced, various kinds of sealing stoppers with various effective diameters of the ribs can be used.

According to an advantageous embodiment of the invention, the outer diameter of the sealing stopper is not more than 15 mm. In this way, it is possible to always seal the cervix of individuals who have not given birth and/or have given birth several times in a secure manner. Incidentally, depending on the expected dimensions of the cervical passage, it is possible to provide sealing stoppers of different diameter in order to ensure optimum sealing with minimal inconvenience to the individual to be treated/examined.

Such ribs may be produced in such a manner that they form part of the sealing stopper. All this may be produced in a simple manner by injection-molding, for example, and be designed as a disposable product.

According to a further advantageous embodiment of the invention, the sealing stopper is provided with a stop flange which abuts the external ostium or becomes wedged at the entrance.

According to a further advantageous embodiment, a non-return valve is provided which only allows liquid to be displaced in the direction toward the uterus.

The sealing stopper is connected or can be connected to a liquid feed tube. In the first case, such a connection may be permanent, so that the sealing stopper can be introduced into the cervix together with the liquid feed tube. However, it is also possible to design all this in such a manner that it can be coupled, which makes it possible to use existing components. However, in the latter case, it is necessary to provide special return means in order to be able to remove the stopper from the cervix. These return means may comprise a piece of string.

Of course it is also possible to effect coupling and disconnection at the interface of liquid feed tube and liquid-dispensing device. In that case the liquid feed tube can be used as return means. In both cases, i.e. wherein there is disconnection of the liquid dispensing device and the sealing stopper a non return valve should be provided to prevent evacuation of the uterus.

According to a further aspect of the invention, a gel is used, if desired in combination with a contrast medium, in order to enlarge the uterus. The passage for liquid in the sealing stopper is designed accordingly.

The present invention relates to an assembly, comprising a sealing stopper, a liquid dispensing device and a feed tube connecting this liquid-dispensing device to this sealing stopper. A liquid-dispensing device of this type may comprise a simple syringe-type structure which is filled beforehand with gel. The assembly may be supplied in a sealed state (sterilized) to the medic who uses it and the entire device can be disposed of after the gel has been introduced and the treatment/examination has taken place.

The invention also relates to a method for enlarging the uterus by introducing liquid therein using a liquid feed line and stopper, this stopper being provided in the cervix to seal it.

### BRIEF DESCRIPTION OF THE DRAWINGS

The invention will be explained in more detail below with reference to the exemplary embodiments shown in the drawing, in which:

FIG. 1 shows a first embodiment of the assembly according to the invention;

FIGS. 2a, b show a detailed view of two variants of the sealing stopper according to the invention,

FIG. 3 shows the assembly according to the invention introduced in the cervix of an individual to be treated/examined and

FIG. 4a-d show a further embodiment of the assembly according to the invention during introduction and removal thereof.

### DETAILED DESCRIPTION OF THE INVENTION

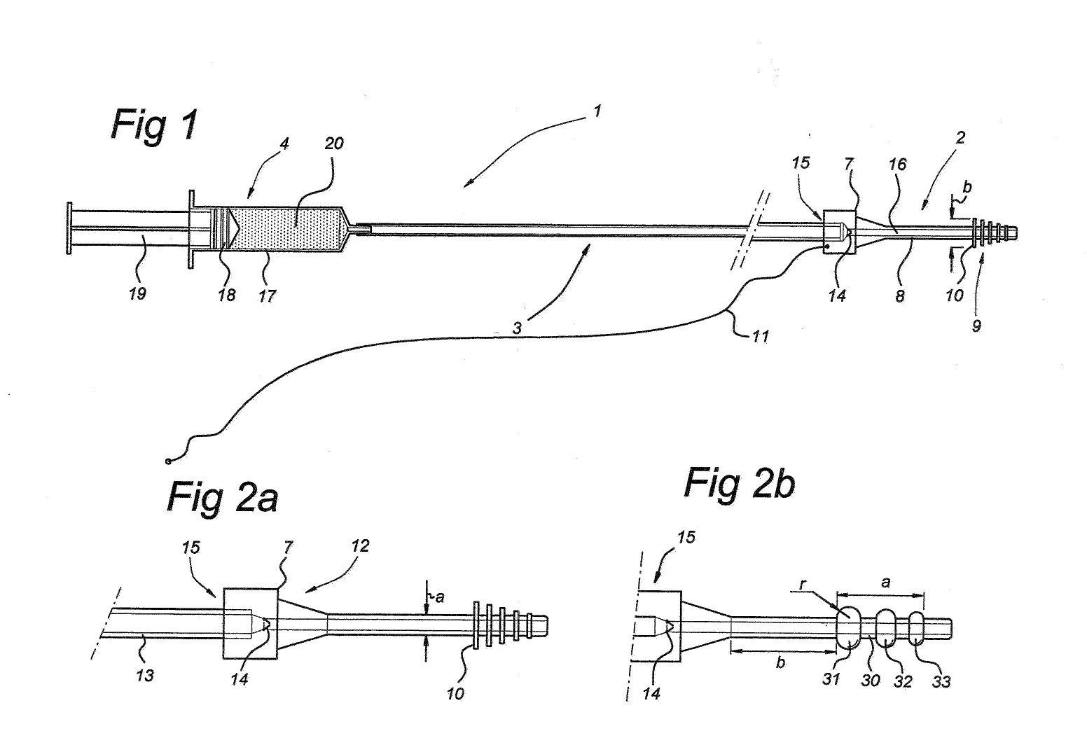

In FIG. 1, the assembly according to the present invention is denoted overall by reference numeral 1. It consists of a sealing stopper 2, a connecting hose 3 connected there to, and a syringe 4. The connecting hose 3 is accommodated in the socket 15 of a thickened part 7 of the sealing stopper 2 in a slightly clamping manner. This thickened part 7 simultaneously serves as a stop. A tube 8 is connected to the latter. A liquid supply duct 16 extends through the entire assembly. Reference numeral 14 denotes a non-return valve. Near its free end, the tube 8 is provided with flexible ribs 10. The tube 8 and ribs 10 form the parts to be introduced and are denoted by reference numeral 9. The external diameter b of the flexible ribs 10 is preferably less than 15 mm.

Connecting hose 3 can be coupled to a syringe-type structure 4 which consists of a piston 18 connected to a handle 19 which piston is guided in a cylinder 17 containing gel 20.

Stop 7 is connected to a piece of string 11.

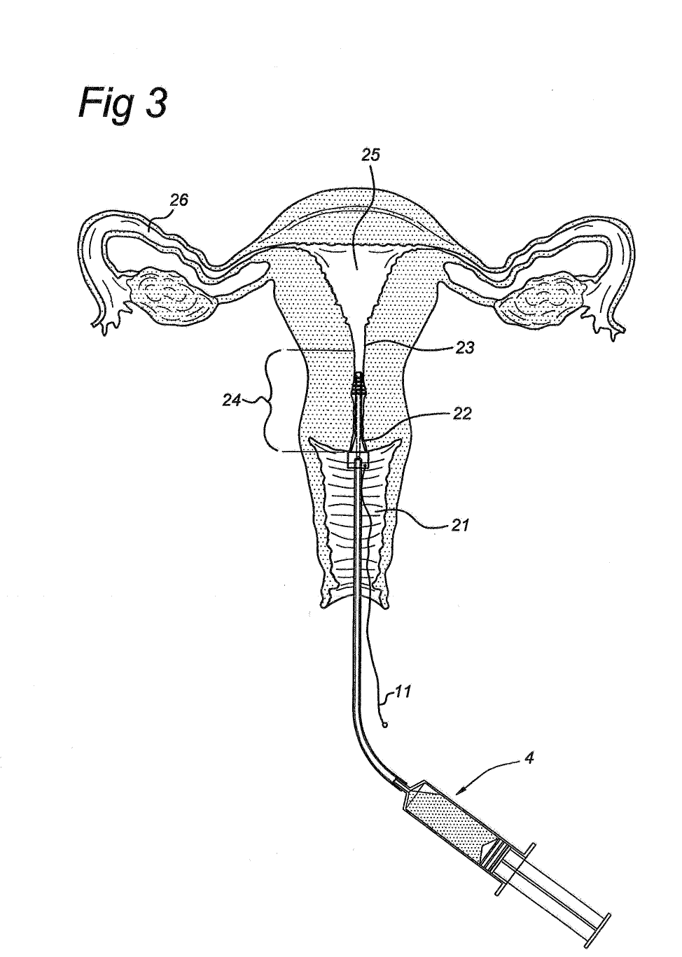

FIG. 2a shows a first variant of the structure described above in which the connecting tube is denoted by reference numeral 13 and fixedly connected to the socket 15 in the stop 7 of the sealing stopper. The external diameter of the tube is denoted by a and is less than 10 mm.

FIG. 2b shows another variant, the ribs being denoted by reference numerals 31-33. Similarly to the variant described above, the diameter of the ribs decreases in the direction of the free end of the core 30. The ribs 31-33 are of spherical design in this case. In this example, the rib 31 has a diameter of approximately 6 mm, rib 32 has a diameter of approximately 5 mm and rib 33 has a diameter of approximately 4 mm. The total distance a over which the ribs extend is approximately 3 cm. The distance b up to the inlet of socket 15 is approximately 3.5 cm.

The ribs 31-33 can be integrally formed with the core 30. It is also possible to use another, for example a softer, material to make it in order to achieve the desired flexibility. By way of example, the use of foam material may be mentioned.

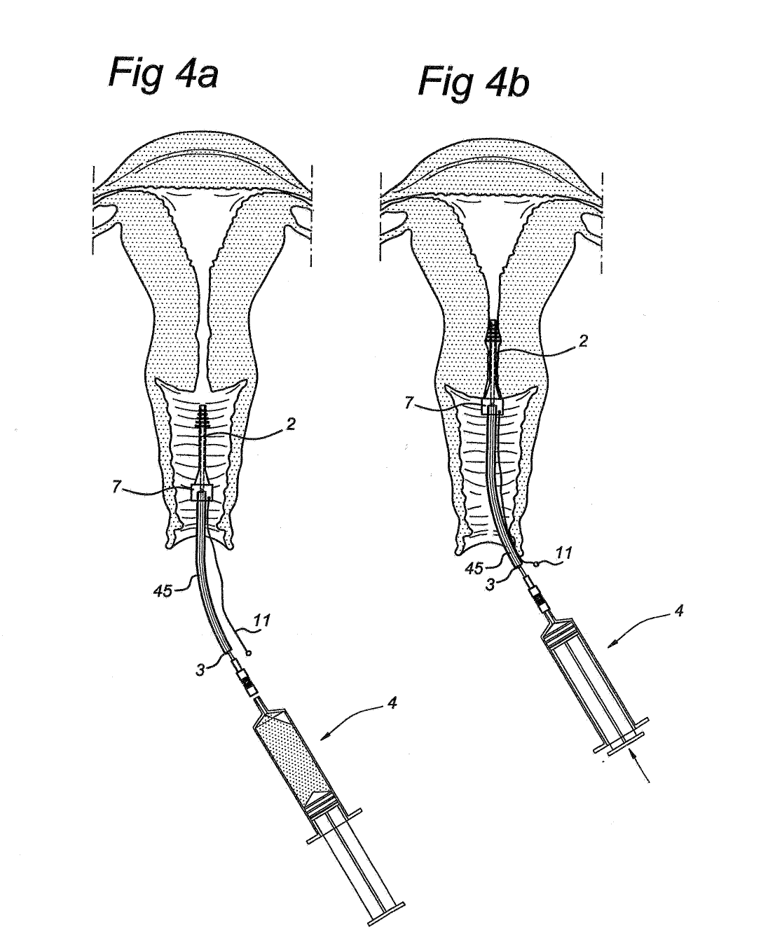

In FIG. 3, the abovementioned assembly 1 is shown fitted in the cervix of an individual to be treated/examined. The syringe 4 is located outside this individual. Connecting hose 3 is introduced via the vagina until it reaches the external ostium. The stop 7 of the sealing stopper 2 moves up to the entrance of the external ostium or is pushed slightly into it and is wedged in to some extent by means of the ribs 10. This clamping force is relatively small as the sealing stopper 2 only has to be prevented from being able to come out easily. Then, using operating handle 19, gel 20 is pressed out of the cylinder 17 into the uterus of individual to be treated/examined via connecting tube 3 and non-return valve 14. Once this operation has finished, the tube 3 can be uncoupled from the sealing stopper 2 by applying a small force, optionally in combination with the introduction of detaining means (also manual). In such a situation, the tube 3 can be removed from the vagina and only the piece of string 11 protrudes from the body of the individual to be treated/examined. Due to the presence of the non-return valve 14, the gel cannot flow back and the uterus remains enlarged as a result of the introduction of the gel. This valve may be designed in any way known in the prior art and, for example, comprise a ball valve. The gel is preferably a medically acceptable gel to which (pain-killing) additives can be added. Once the treatment/examination has finished, the sealing stopper 2 according to the invention can be removed in a simple manner by pulling on the piece of string 11. In principle, it is also possible to remove the sealing stopper 2 using a pair of pliers.

If the assembly is used in combination with the sealing stopper according to FIG. 2, no piece of string 11 will be present and the connecting hose 13 will be permanently connected to the sealing stopper 12. This means that the connecting hose now protrudes from the body of the individual to be treated/examined instead of the piece of string and the connecting hose can be removed in this manner.

In both cases, the individual to be treated/examined is able to move about freely after the introduction of the sealing stopper and the gel, so that it is possible, for example, to carry out this first part of the treatment/examination at an outpatients' department and to carry out the next part of the treatment/examination with the enlarged uterus in a completely different location. The seal is guaranteed to be completely tight, so that no uncomfortable situations arise.

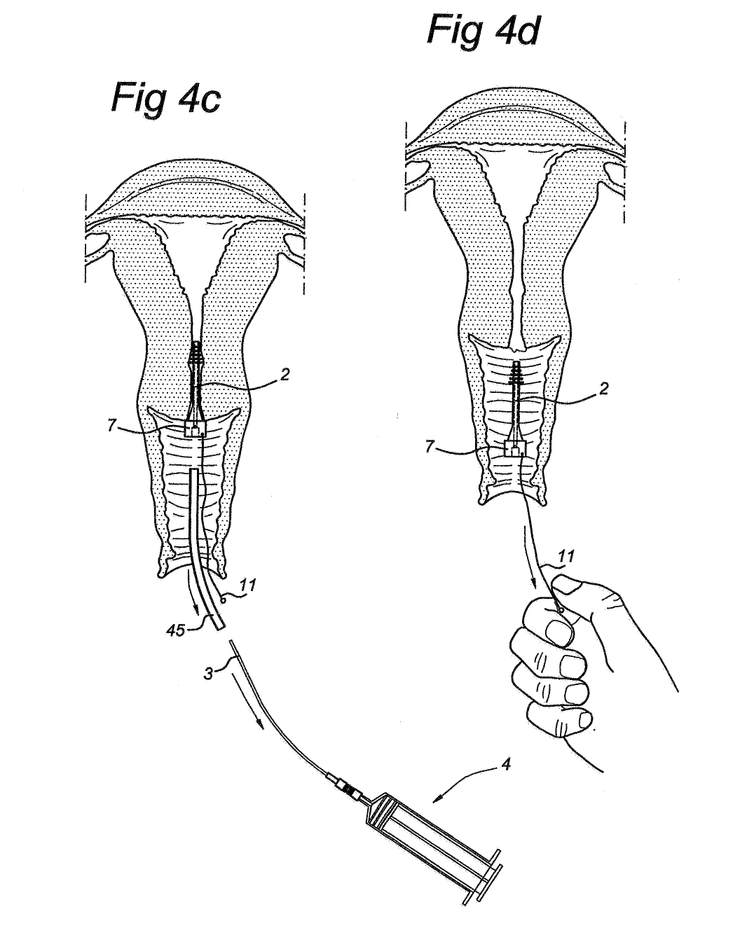

FIG. 4a-d shows a further embodiment of the invention substantially corresponding with the embodiment shown in the previous figures. Components corresponding with components shown in these previous figures have been given the same reference numbers. Also in this case injection syringe 4 is connected to stopper 2 through a conduit 3. Syringe 4, conduit 3 and stopper 2 are separate items. Stopper 2 is provided with return means 11. FIG. 4a shows introduction of the assembly according to the invention. In the kit as provided, syringe 4 is separate from a sub-assembly comprising conduit 3 and stopper 2. A free sliding sleeve 45 is inserted over conduit 3 and abuts against the thickened part 7 of sealing stopper 2.

Before or after connecting syringe 4 to conduit 3 the assembly is entered in the human body as shown in FIG. 4b. Consequently the contents of the syringe is transferred to the related cavity. Preferably this is effected at the presence of a physician and more particular a gynecologist.

According to the invention the uterus of the female patient should be re-examined. According to the invention the invention sealing of the stopper relative to the uterus is such that no liquid escapes, i.e. the extended condition of the uterus will be maintained. The patient is now transported to an examination location where for example echoscopy can be effected without the gynaecologist being present. To reduce hindrance for the patient as much as possible it is desirable to removed syringe 4. It is possible to simply disconnect syringe 4 from conduit 3.

However according to this embodiment of the invention tube 3 with syringe 4 is removed from sealing stopper 2. In order to prevent displacement of the sealing stopper 2 when a tension force is exerted on tube 3, sleeve 45 as described above is present. The physician with one hand pushes conduit 3 in outward direction whilst with his other hand an equal pushing force is exerted on sealing stopper 2 through tube 45. After disconnection takes place between the conduit 3 and sealing stopper 2 both the conduit and sleeve 45 can be easily removed as is shown in FIG. 4c.

After examination through the presence of our wire 11, simple pull thereon will remove the stopper from the human body.

In this embodiment a check valve is provided in sealing stopper 2. In an alternative embodiment wherein both the sealing stopper and the conduit 3 remain with the human to be examined such a check valve could be present in tube 3.

Permanent and stable enlargement of the uterus is important for a variety of treatments. As indicated above, echoscopy is one example thereof, more in particular three-dimensional echoscopy, where assembly of the image is relatively slow. Other examination techniques, such as CT scans or MRI techniques (optionally in 3D) are possible.

The thickness of the gel may be adapted to the treatment. Preferably, this gel is a substance which has a relatively high viscosity at higher (body) temperature(s) and a relatively low viscosity at lower temperatures. The amount Of gel used may be relatively small (for example approximately 10 ml).

The assembly according to the present invention is preferably supplied in assembled form, so that uncoupling of the liquid supply device only takes place after the sealing stopper has been put into place and the liquid has been introduced. Then, the patient can be examined in another location and subsequently the sealing stopper can be removed.

Although the invention has been described above using a preferred embodiment, it will be understood that numerous modifications can be made thereto, based on the idea of sealing the uterus for various treatments/examinations. Such modifications are within the scope of the appended claims.

1. Assembly comprising a sealing stopper to be introduced into the cervix in order to seal it, provided with a passage for liquid embodied for the passage of a gel comprising a core and flexible cervix-engagement means arranged around the core, a liquid-dispensing device filled with a gel and a feed tube connecting this liquid-dispensing device to said sealing stopper.

2. Assembly according to claim 1, wherein coupling means are provided between the liquid-dispensing device and the sealing stopper.

3. Assembly according to claim 1 wherein the feed tube can be disconnected from the sealing stopper.

4. Assembly according to claim 1 wherein said cervix-engagement means comprising ribs spaced from one another.

5. Assembly as claimed in claim 4, wherein said ribs comprise spherical parts.

6. Assembly as claimed in claim 1, comprising a non-return valve which only allows liquid to be displaced in the direction toward the uterus.

7. Assembly as claimed in claim 1, wherein said sealing stopper comprises a stop flange.

8. Assembly as claimed in claim 1, comprising return means connected to the stopper.

9. Assembly as claimed in claim 8, wherein said return means comprise a piece of string.

10. Assembly according to claim 1, wherein said liquid-dispensing device comprises a gel-dispensing device with a piston/cylinder.

11. Method for enlarging the uterus by introducing a liquid using a liquid feed line and stopper, characterized in that this stopper is arranged in the cervix in a scaling manner.

12. Method as claimed in claim 1, wherein the introduction of the stopper followed by the introduction of liquid is carried out in one location and the individual to be treated/examined then moves to another room, at some distance from the introduction location, for the treatment/examination.
